(19) United States
(12) Patent Application Publication
(10) Pub. No.: US 2011/0125072 A1
(43) Pub. Date: May 26, 2011

(54) Posture improvement device

(76) Inventor: John Michael Lane, New York, NY (US)

(21) Appl. No.: 12/954,270

(22) Filed: Nov. 24, 2010

Related U.S. Application Data
(60) Provisional application No. 61/263,825, filed on Nov. 24, 2009.

(51) Int. Cl. A61F 5/00

(52) U.S. Cl. 602/19

(57) ABSTRACT

The present invention is a posture improvement device that includes an adjustable flexible tension band with two ends that extends across a user's back to provide posture support, an adjustable arm band attached to each adjustable flexible tension band end to be secured on the user's upper arm, a first adjustable fastener disposed on each adjustable arm band to secure and accommodate different sizes of the users' upper arms to provide comfort and proper fitting and a second adjustable fastener disposed on the adjustable flexible tension band to secure and accommodate different sizes of users' back widths to provide comfort and proper fitting. The invention also includes a method for using the posture improvement device.

This application claims priority to U.S. Provisional Application 61/263,825 filed on Nov. 24, 2009, the entire disclosure of which is incorporated by reference.

### TECHNICAL FIELD & BACKGROUND

The present invention generally relates to a posture device. More specifically, the invention is a posture improvement device.

It is an object of the invention to provide a posture improvement device that is adjustably attached to a user's upper arms rather than wrapping around or over a user's shoulders.

It is an object of the invention to provide a posture improvement device that utilizes a user's back muscles to pull the user's shoulders back for improving posture.

It is an object of the invention to provide a posture improvement device that it is made of durable material, which also provides cushioning for comfortable usage.

It is an object of the invention to provide a posture improvement device that can be worn inside or outside of a user's clothing or inside and outside a user's clothing at the same time.

What is needed is a posture improvement device that is adjustably attached to a user's upper arms rather than wrapping around or over a user's shoulders, that utilizes a user's back muscles to pull the user's shoulders back, that it is made of durable material, which also provides cushioning for comfortable use and that can be worn inside or outside of a user's clothing or inside and outside a user's clothing at the same time.

### BRIEF DESCRIPTION OF THE DRAWINGS

The present invention will be described by way of exemplary embodiments, but not limitations, illustrated in the accompanying drawing in which like references denote similar elements, and in which:

FIG. 1 illustrates a rear side environmental perspective view of a posture correcting device with an unclothed user, in accordance with one embodiment of the present invention.

FIG. 2 illustrates a rear side environmental perspective view of a posture correcting device with a clothed user, in accordance with one embodiment of the present invention.

FIG. 3 illustrates a flow chart of a method for using a posture improvement device, in accordance with one embodiment of the present invention.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

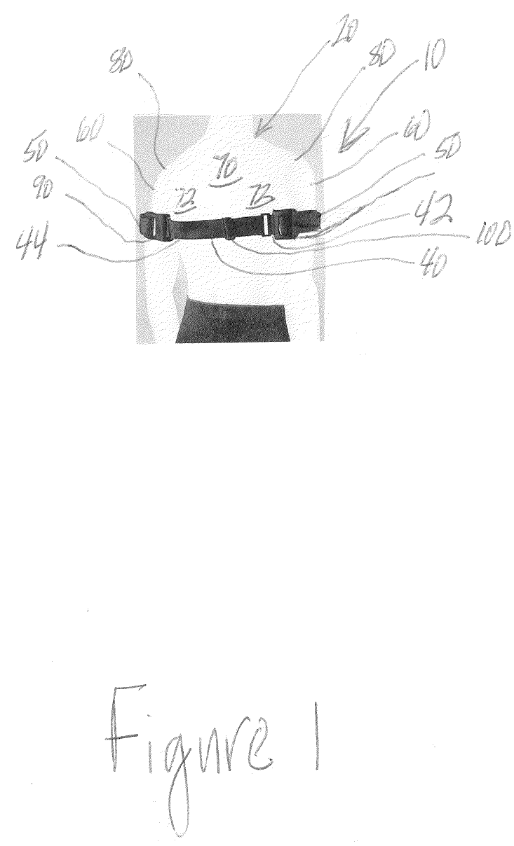

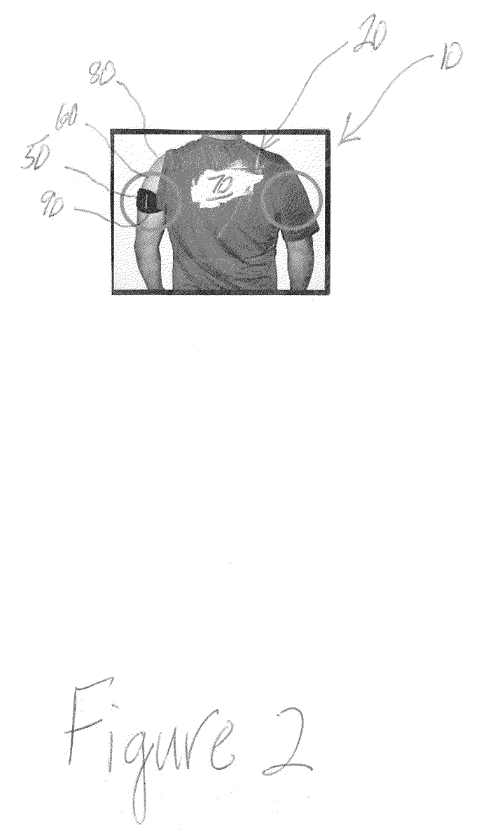

FIG. 1 illustrates a rear side environmental perspective view of a posture improvement device 10 with an unclothed user 20, in accordance with one embodiment of the present invention. FIG. 2 illustrates a rear side environmental perspective view of a posture correcting device 10 with a clothed user 20, in accordance with one embodiment of the present invention.

The posture improvement device 10 is designed to be worn for short periods of time, rather than continually, as with some posture improvement devices that are well known in the art. The posture improvement device 10 achieves the desired result of improved posture by providing a gentle reminder to a user 20 to keep their shoulders back and back straight, utilizing the power of the user's 20 own back muscles. When a user 20 is in a correct posture position, there is little tension on the adjustable flexible tension band 40 of the posture improvement device 10. When a user 20 begins to slouch, the tension from the posture improvement device 10 begins to increase, which reminds the user 20 to resume correct posture. The posture improvement device 10 trains and strengthens the back muscles of a user 20 also, which is vital for good posture.

The components and design of the posture improvement device 10 are straightforward. The posture improvement device 10 is comprised of two adjustable arm bands 50 that are disposed on each end 42, 44 of an adjustable flexible tension band 40. The adjustable arm bands 50 are secured around the upper arms 60 of a user 20 while the attached adjustable flexible tension band 40 extends across the user's back 70 and provides support and posture improvement when the user 20 begins to slouch. The tension provided by the adjustable flexible tension band 40 reminds the user 20 to pull his or her shoulders 80 back, putting the user 20 in an improved posture position. The posture improvement device 10 is different from other posture improvement devices and posture improvement braces, in that it attaches to the user's upper arms 60, rather than wrapping around or over the user's shoulders 80. The posture improvement device 10 also utilizes the user's back 70 and specifically their back muscles 72 to pull the user's shoulders 80 back. Some posture improvement devices that are well known in the art depend on tension created by the posture improvement device 10 to pull back a user's shoulders 80, which actually can cause the user's back muscles to weaken. The posture improvement device 10 is made of durable Neoprene™ material, which provides cushioning for comfortable use. The posture improvement device 10 can also be made of other durable materials that are well known in the art.

The posture improvement device 10 can be worn inside or outside of a user's clothes or inside and outside of a user's clothes at the same time, as is shown in FIG. 2. The user 20 can place one adjustable arm band 50 on each of his or her upper arms 60, with the adjustable flexible tension band 40 running across the user's back 70. The posture improvement device 10 provides light tension across a user's back 70, which increases if the user 20 begins to slouch or exhibit poor posture. The adjustable arm bands 50 utilize a first adjustable fastener 90 disposed on each adjustable arm band 50 to accommodate different sizes of users' upper arms 60 and the adjustable flexible tension band 40 to accommodate the different sizes of users' back widths so that the posture improvement device 10 can be used by users of all sizes, body types and sexes. Once a user 20 has placed an adjustable arm band 50 on each upper arm 60, the user 20 can easily adjust the posture improvement device 10 for comfort and proper fitting by utilizing the first adjustable fastener 90 on each adjustable arm band 50. The user 20 can then adjust the adjustable flexible tension band 40 using a second adjustable fastener 100 provided on the adjustable flexible tension band 40 for comfort and proper fitting. Both the first adjustable fasteners 90 and second adjustable fasteners 100 are slidably adjustable, where a user 20 can slide the adjustable fasteners along the adjustable arm band 50 or adjustable flexible tension band 40 for a proper comfortable fit, but any type of adjustable fastener well known in the art can be used with the posture improvement device 10.

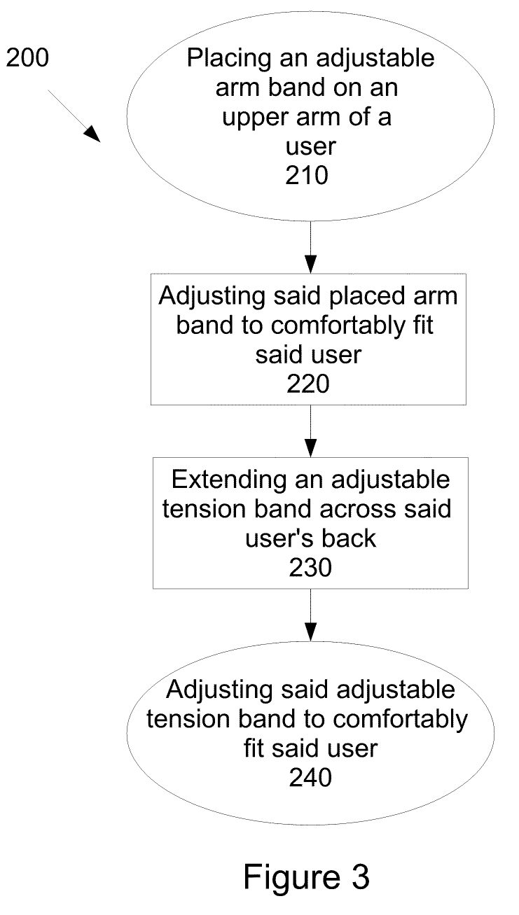

FIG. 3 illustrates a flow chart of a method of using a posture improvement device 200, in accordance with one embodiment of the present invention. The method of using a posture improvement device 200 includes the steps of placing an adjustable arm band around each upper arm of a user 210, adjusting the placed adjustable arm band on the upper arms of the user to comfortably fit the user 220, extending an adjustable flexible tension band disposed between each adjustable armband across the user's back 230 and adjusting the disposed adjustable flexible tension band to comfortably fit the user 240.

As previously indicated in the FIGS. 1 and 2 descriptions, the adjustable arm bands are adjusted using a first adjustment fastener disposed on each adjustable arm band to comfortably fit the upper arms of the user. The adjustable flexible tension band is similarly adjusted using a second adjustment fastener to comfortably fit the back width of the user. Both the first and second adjustment fasteners are slidably adjustable, but any adjustment fastener that is well known in the art can be used. The posture improvement device can be worn inside or outside of a user's clothes or both inside and outside of a user's clothes at the same time.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

1. A posture improvement device, comprising:
an adjustable flexible tension band with two ends that extends across a user's back to provide posture support;
an adjustable arm band attached to each said adjustable flexible tension band end to be secured on said user's upper arm;
a first adjustable fastener disposed on each said adjustable arm band to secure and accommodate different sizes of said users' upper arms to provide comfort and proper fitting; and
a second adjustable fastener disposed on said adjustable flexible tension band to secure and accommodate different sizes of said users' back widths to provide comfort and proper fitting.

2. The device according to claim 1, wherein said first adjustable fastener is slidably adjustable along said adjustable arm band.

3. The device according to claim 1, wherein said second adjustable fastener is slidably adjustable along said adjustable flexible tension band.

4. The device according to claim 1, wherein said posture improvement device is designed to be worn for short periods of time.

5. The device according to claim 1, wherein said posture improvement device can be worn inside or outside of said user's clothes.

6. The device according to claim 1, wherein said posture improvement device can be worn inside and outside of said user's clothes at the same time.

7. The device according to claim 1, wherein said second adjustable fastener is slidably adjustable along said adjustable flexible tension band.

8. The device according to claim 1, wherein said posture improvement device is made of neoprene.

9. A posture improvement device, comprising:
two adjustable arm bands;
a tension band connecting said two adjustable arm bands;
a first adjustable fastener disposed on each said adjustable arm band; and
a second adjustable, fastener disposed on said adjustable tension band.

10. The device according to claim 9, wherein said first adjustable fastener is adjustable along said adjustable arm band and said second adjustable, fastener is adjustable along said adjustable flexible tension band.

11. The device according to claim 9, wherein said posture improvement device is made of neoprene.

12. A method for using a posture improvement device, comprising the steps of:
placing an adjustable arm band around each upper arm of a user;
adjusting said placed adjustable arm band on the upper arms of said user to comfortably fit said user;
extending an adjustable flexible tension band disposed between each said adjustable arm band across said user's back; and
adjusting said disposed adjustable flexible tension band to comfortably fit said user.

13. The method according to claim 12, wherein said placed adjustable arm band is adjusted with a first adjustable fastener.

14. The method according to claim 13, wherein said first adjustable fastener is slidably adjustable along said adjustable arm band.

15. The method according to claim 12, wherein said extended adjustable flexible tension band is adjusted with a second adjustable fastener.

16. The method according to claim 15, wherein said second adjustable fastener is slidably adjustable along said adjustable flexible tension band.

17. The method according to claim 12, wherein said posture improvement device is designed to be worn for short periods of time.

18. The device according to claim 12, wherein said posture improvement device can be worn inside or outside of said user's clothes.

19. The device according to claim 12, wherein said posture improvement device can be worn inside and outside of said user's clothes at the same time.

20. The device according to claim 12, wherein said posture improvement device is made of neoprene.
